(19) United States
(12) Patent Application Publication
(10) Pub. No.: US 2022/0061821 A1
(43) Pub. Date: Mar. 3, 2022

(54) DEVICE, KIT AND METHOD FOR COLLECTING ENDOCERVICAL AND VAGINAL SAMPLES

(71) Applicant: Cradle Genomics, Inc., San Diego, CA (US)

(72) Inventors: Tristan Orpin, San Diego, CA (US); Richard Shippy, San Diego, CA (US); Silvia Gravina, San Diego, CA (US)

(21) Appl. No.: 17/411,535

(22) Filed: Aug. 25, 2021

Related U.S. Application Data
(60) Provisional application No. 63/070,686, filed on Aug. 26, 2020.

(51) Int. Cl. A61B 10/00

(52) U.S. Cl. CPC A61B 10/0045; A61B 2010/0074

(57) ABSTRACT

The present disclosure provides a device for collection of an endocervical or vaginal sample, as well as a method of using the device to collect a sample a subject and perform an assay. The collection device includes a resiliently deformable ring, a flexible membrane coupled to the ring which defines a collection reservoir, and a retrieval element configured for removal of the device.

### CROSS REFERENCE TO RELATED APPLICATIONS

This application claims the benefit of U.S. Provisional Patent Application No. 63/070,686, filed on Aug. 26, 2020, which is herein incorporated by reference in its entirety.

### BACKGROUND OF THE INVENTION

### Field of the Invention

The present invention relates generally to diagnostics and more specifically to a device and method for collecting an endocervical or vaginal sample from a subject and subsequent analysis of the sample for diagnostic purposes.

### Background Information

The field of medicine relies heavily on investigative testing performed on various compositions of biological material. Testing biological samples, such as bodily fluids containing cells or other analytes, often involves collecting a biological sample and exposing the sample to a diagnostic testing assay.

An assay generally involves bringing a portion of the sample into contact with an assay in order to qualitatively assess or measure the presence and/or amount of an anticipated analyte in the sample. Many such test assays are known to detect for the presence of a specific cell, chemical, nucleic acid, hormone, or other material in a biological sample, including a biological fluid sample, such as blood, urine, saliva, cervical fluid and the like. Such tests have been designed, manufactured, produced, and marketed for use in such fields as home, industrial, veterinary, and occupational testing, among others.

In some instances, a biological sample is collected by a user in an at-home setting which is remote to a laboratory testing facility where an assay is performed using the collected sample. In such instances, the sample is collected by the user and then transported to testing facility in a sample container. Often, sample collection by the user may be difficult or uncomfortable particularly in instances where the sample must be collected using an invasive device as is the case for collection of endocervical fluid.

Accordingly, a need for an improved device for endocervical sample collection which is easy, convenient and comfortable for the user exists.

### SUMMARY OF THE INVENTION

In one embodiment, the disclosure provides a device for collecting an endocervical or vaginal sample. The device includes: a) a resiliently deformable ring having a central opening, wherein the ring has a thickness defined by an outer circumference surface and an inner circumference surface, and a height defined by an upper rim and a lower rim; b) a flexible membrane coupled to the ring and covering the central opening thereby defining a collection reservoir; and c) a retrieval element configured for removal of the device by a user from the vaginal canal.

The disclosure also provides a kit which includes a device of the invention, a storage container for transport of the device containing a collected sample, and optionally an applicator configured to deploy the device within the vaginal canal.

In another embodiment, the disclosure provides a method of using the device of the disclosure which includes: a) disposing the collection device of the invention at a location proximal to an external os of a uterine cervix or anywhere within the vaginal canal of a subject; b) collecting an endocervical or vaginal sample in the collection reservoir while the device is located proximal to the external os or anywhere within the vaginal canal; c) removing the device containing the endocervical or vaginal sample from the subject; and optionally d) conducting an assay to detect an analyte present in the endocervical or vaginal sample.

In still another embodiment, the disclosure provides a method of using the device of the invention which includes conducting an assay to detect an analyte present in an endocervical or vaginal sample from a subject, wherein the sample is collected into the device of the invention disposed at a location proximal to an external os of a uterine cervix or anywhere within the vaginal canal of the subject.

### BRIEF DESCRIPTION OF THE FIGURES

FIG. 1 is a perspective view of a sample collection device in one embodiment of the invention.

FIG. 2 is a side view of the sample collection device of FIG. 1 showing the retrieval element in one embodiment of the invention.

FIG. 3 is a perspective view of a sample collection device in one embodiment of the invention.

FIG. 4 is a side view of the sample collection device of FIG. 3 showing the retrieval element in one embodiment of the invention.

FIG. 5 is a perspective view of a sample collection device in one embodiment of the invention.

FIG. 6 is a bottom perspective view of the sample collection device of FIG. 5.

FIG. 7 is a top view of the sample collection device of FIG. 5.

FIG. 8 is a bottom view of the sample collection device of FIG. 5.

FIG. 9 is a perspective view of a sample collection device in one embodiment of the invention.

FIG. 10 is a top view of the sample collection device of FIG. 9.

FIG. 11 is an expanded view of the sample collection device of FIG. 9 showing the attachment of the retrieval element to the ring.

FIG. 12 is a top view of the sample collection device of FIG. 9.

FIG. 13 is a bottom view of the sample collection device of FIG. 9.

FIG. 14 is a perspective view of a sample collection device in one embodiment of the invention.

FIG. 15 is a bottom perspective view of the sample collection device of FIG. 14.

FIG. 16 is a perspective view of a sample collection device in one embodiment of the invention showing the retrieval element attached to the ring such that it extends from the ring at two different angles.

FIG. 17 is a perspective view of a sample collection device in one embodiment of the invention showing the retrieval element attached to the ring such that it extends perpendicular to the ring.

FIG. 18 is a perspective view of a sample collection device in one embodiment of the invention.

FIG. 19 is a perspective view of a sample collection device in one embodiment of the invention.

FIG. 20 is a perspective view of a sample collection device in one embodiment of the invention.

FIG. 21 is a side view of a sample collection device in one embodiment of the invention.

FIG. 22 is a side view of a sample collection device in one embodiment of the invention.

FIG. 23 is a side view of a sample collection device in one embodiment of the invention.

### DETAILED DESCRIPTION OF THE INVENTION

The present disclosure is based on an innovative device for collection of an endocervical or vaginal sample. The device is operable to be deployed proximal to an external os of a uterine cervix or anywhere within the vaginal canal of a female human subject.

Before the present compositions and methods are described, it is to be understood that this invention is not limited to particular devices, methods and experimental conditions described, as such devices, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Accordingly, in one embodiment, the disclosure provides a device for collecting an endocervical sample. With reference to the Figures, the device includes a resiliently deformable ring 10 having a central opening, wherein the ring 10 has a thickness defined by an outer circumference surface and an inner circumference surface, and a height defined by an upper rim and a lower rim. The device further includes a flexible membrane coupled to the ring 10 and covering the central opening thereby defining a collection reservoir 20. The device also includes a retrieval element 30 to simplify removal of the device from the vaginal canal by a user to retrieve the collected sample.

In various aspects, the device of the invention has the capability of enabling patient-directed biological self-collections. The device provides ease of use and comfort to the user for collection of endocervical or vaginal samples.

In certain aspects, the ring is composed of a resiliently deformable material. The device may be compressed into an elongated configuration for insertion into the vaginal canal and automatically circularizes to conform to the walls of the vagina as it locates directly below the cervix at the upper end and behind the public bone. In various aspects, the diameter of the ring is between about 4.5 and 8.0 cm, including 4.5, 5.0, 5.5, 6.0, 6.5, 7.0, 7.5 and 8.0 cm. It is sized to expand so that it fits securely in the fornix or another region of the vaginal canal.

The device of the invention is resiliently deformable and composed of one or more resiliently deformable biocompatible materials so long as the materials are substantially non-degradable. Such materials include biocompatible polymers. Biocompatible materials for use in the collection device include, but are not limited to polyethylene (PE), nylon, polyamide, polyether block amides (PEBAX), polyethylene terephthalate (PET), silicone, POC, polypropylene and polyether block PBT. In one aspect, the entire device is composed of the same material. In another aspect, the ring and membrane are formed of different materials.

As illustrated in the Figures, the thin flexible membrane is coupled to the ring 10 and covers the central opening of the ring to form a collection reservoir 20 for collecting fluid. Coupling the membrane to the ring 10 may be accomplished by any number of conventional techniques such that a fluid tight seal between the ring surface and the membrane is formed. Alternatively, the ring 10 and membrane may be formed as a unitary material.

Further, the membrane may be coupled to the ring 10 at a variety of locations to form the collection reservoir 20. For example, the membrane may be coupled to the inner circumference surface at any point between the upper and lower rim. In one aspect, the membrane is coupled to the inner circumference surface at a point oriented toward the lower rim. In another aspect, the membrane is coupled about the circumference of the lower rim or an inner portion of the circumference of the lower rim oriented toward the central opening such that the retrieval element 30 may be coupled to the ring 10 without puncturing or damaging the integrity of the membrane.

The device further includes a retrieval element 30 of any suitable length that allows the device to be easily removed from the vaginal canal by the user to retrieve the collected sample. As illustrated in the Figures, a variety of configurations of the retrieval element 30 are envisioned. In various aspects, the retrieval element 30 is attached to the ring 10 such that the coupling is permanent and allows for removal of the device by a user by solely pulling on the retrieval element 30. Additionally, the retrieval element 30 is coupled to the ring 10 in a manner such that it does not result in risk of the membrane being damaged. The coupling location of the retrieval element 30 may also assist in ensuring the correct orientation (up/down) of the device during insertion.

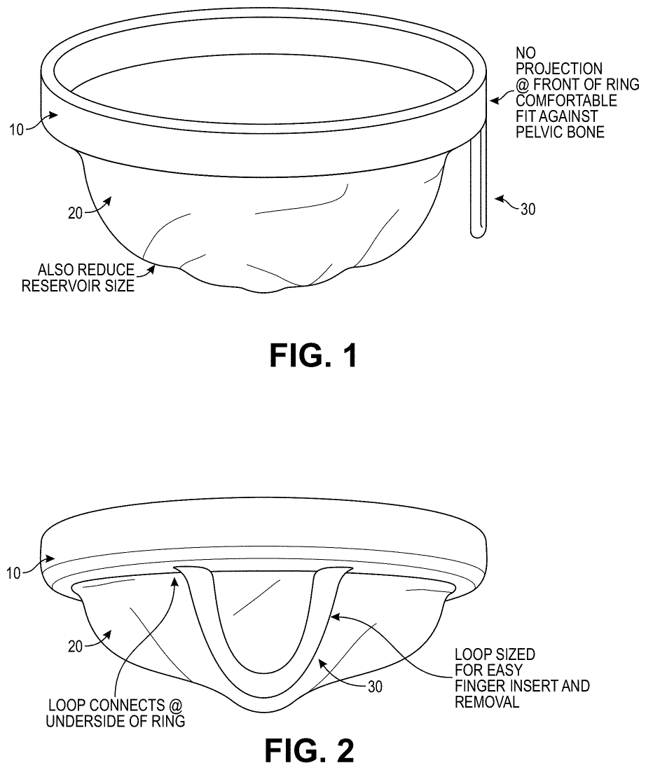

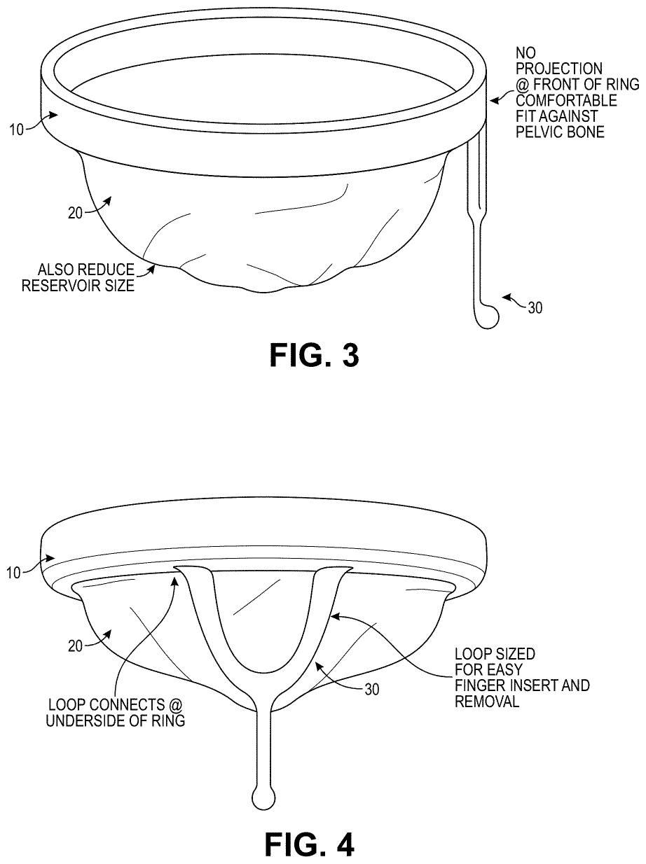

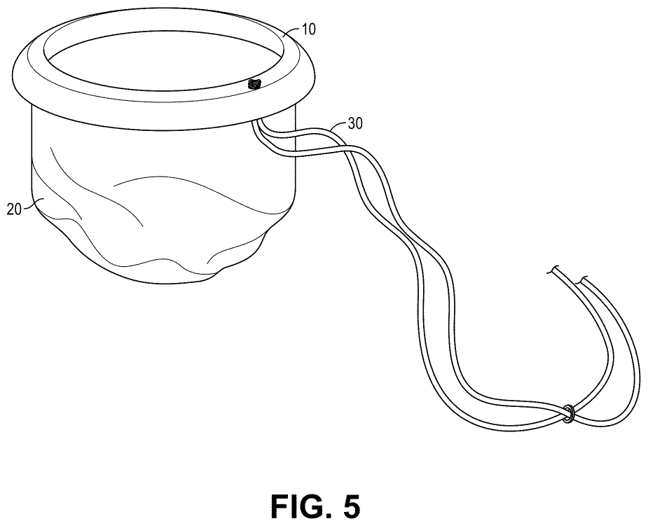

FIGS. 1-4 illustrate an embodiment in which the retrieval element 30 is molded into the ring 10 and extends from the lower rim in a loop type configuration. In various aspects, the retrieval element 30 may be molded into the ring 10 such that there is no projection extending from the outer and/or inner circumference surface of the ring 10. Additionally, the retrieval element 30 can extend from the lower rim at an angle with respect to the outer circumference surface or may extend in a plane parallel to the outer circumference surface as shown in FIGS. 1-4. As shown in FIGS. 3-4, in certain aspects, the retrieval element 30 may include an extension portion extending from the loop such that a user can easily locate and grasp the retrieval element 30 to remove the device from the vaginal canal.

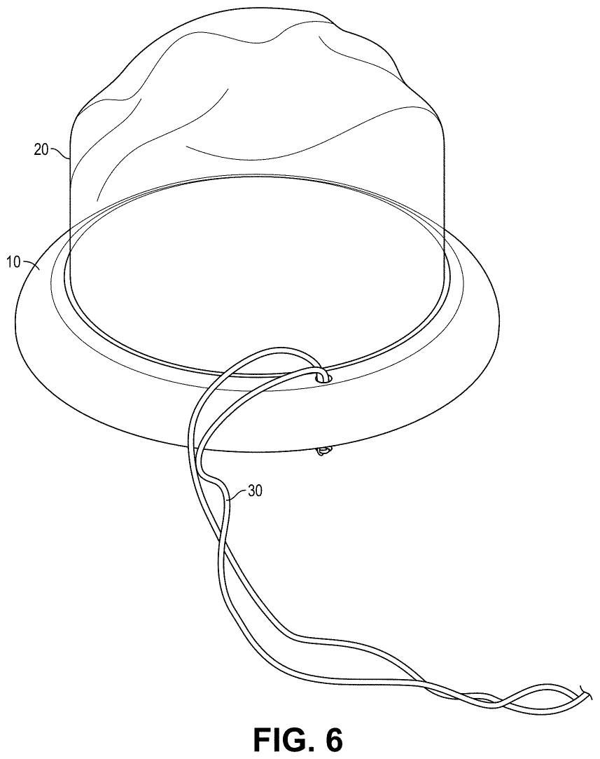

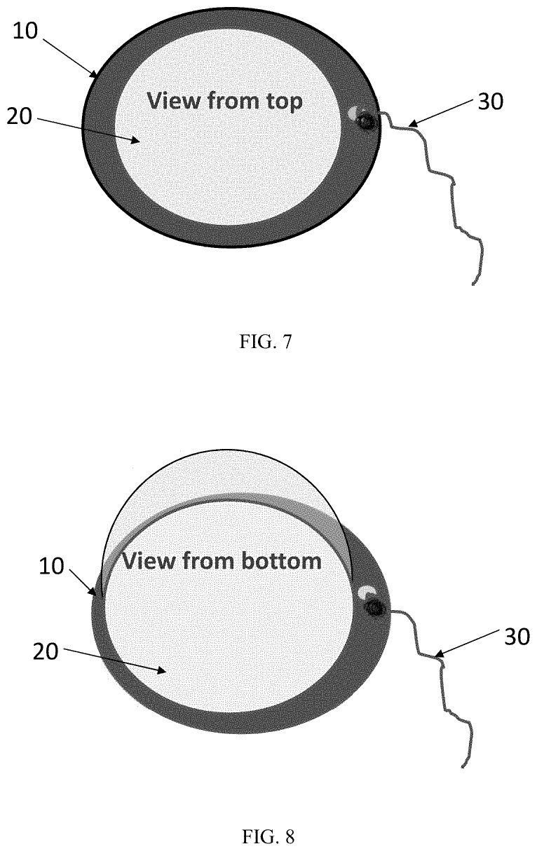

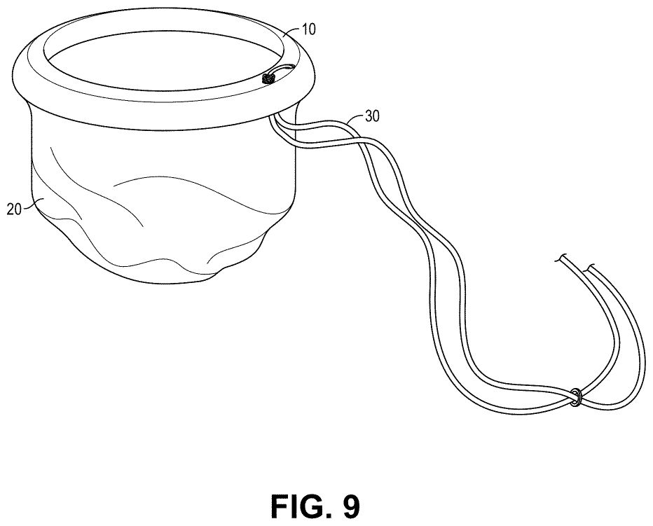

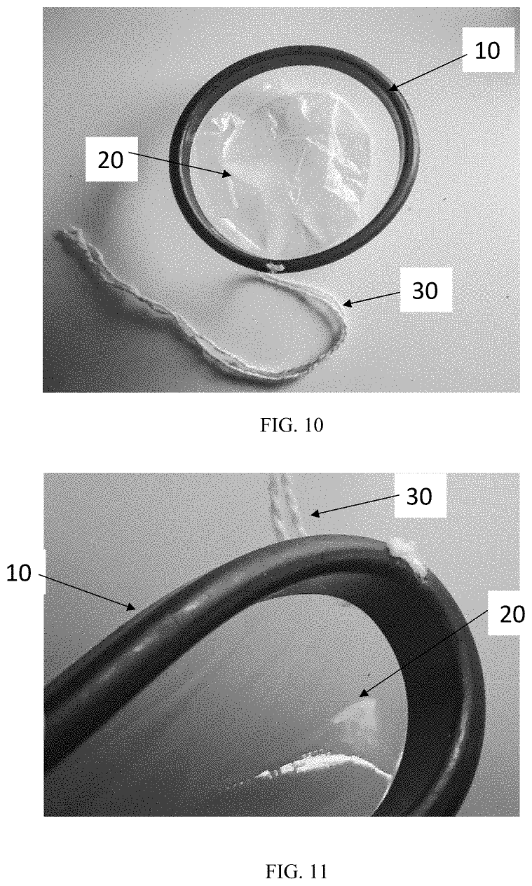

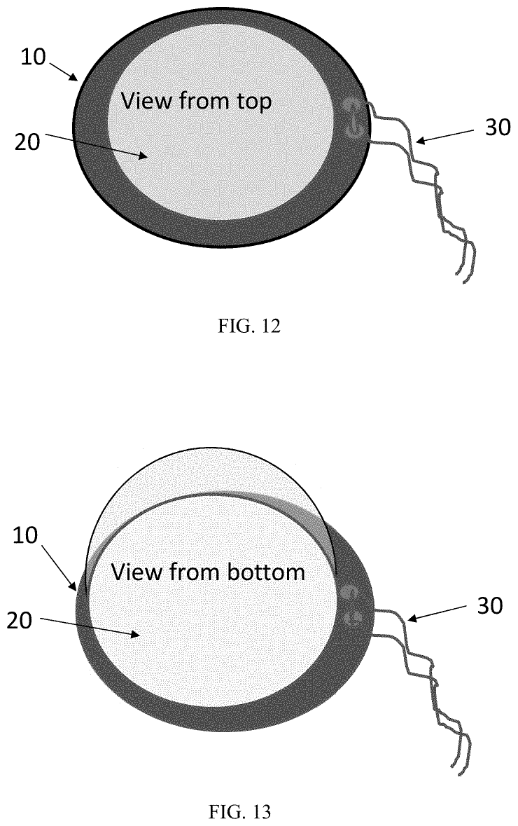

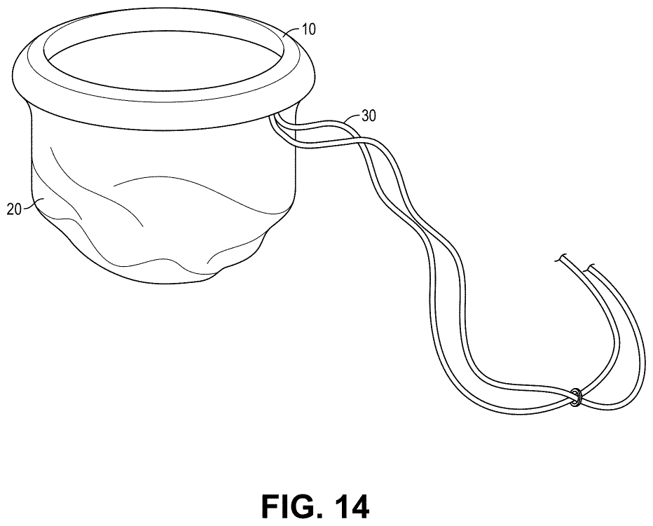

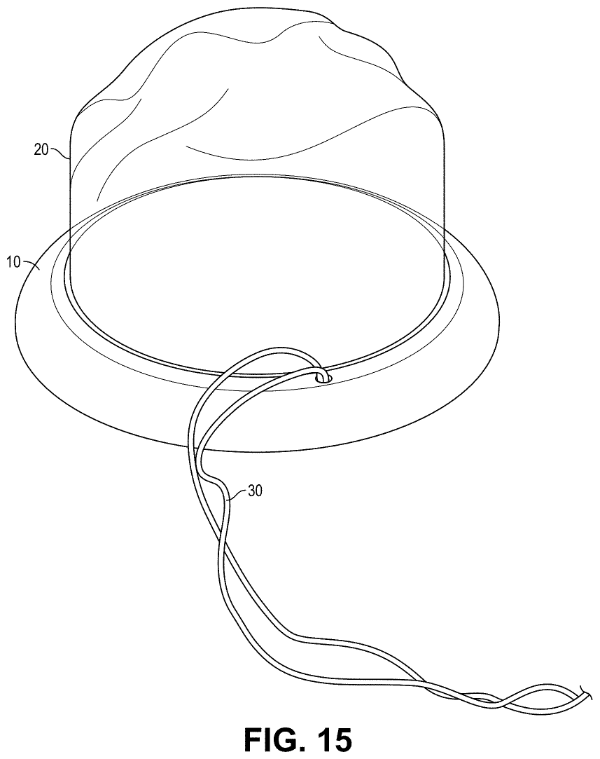

As shown in FIGS. 5-15, the retrieval element 30 may be configured as a string or tether which is attached to the ring 10. The string may include a single strand or multiple strands such that the string may have a loop type configuration. Similarly, a loop may be formed in the single strand, optionally at the terminal portion of the string to facilitate grasping and retrieval of the device from the vaginal canal by the user. Additionally, it will be appreciated that the string may be of any length that facilitates locating and grasping of the string by the user to retrieve the device from the vaginal canal.

As shown in FIGS. 5-15, the string may be attached to the ring 10 in a variety of configurations. In various embodiments, the string is attached to the ring 10 such that the string does not form a protrusion on the outer and/or inner circumference surface of the ring.

FIGS. 5-8 show an embodiment in which the string extends from the lower rim surface of the ring 10 and is attached to the ring 10 via a single through hole extending from the lower rim surface to the upper rim surface. The string is secured in place by one or more knots at the upper and/or lower rim surface.

FIGS. 9-13 show an embodiment in which the string extends from the lower rim surface of the ring 10 and is attached to the ring 10 via two through holes extending from the lower rim surface to the upper rim surface. The string is secured in place by one or more knots at the lower rim surface.

FIGS. 14 and 15 show an embodiment in which the string extends from the lower rim surface of the ring 10 and is attached to the ring 10 about the circumference of the lower rim. In this configuration, no protrusions are formed on the upper rim surface, as well as the outer and inner circumference surfaces of the ring 10.

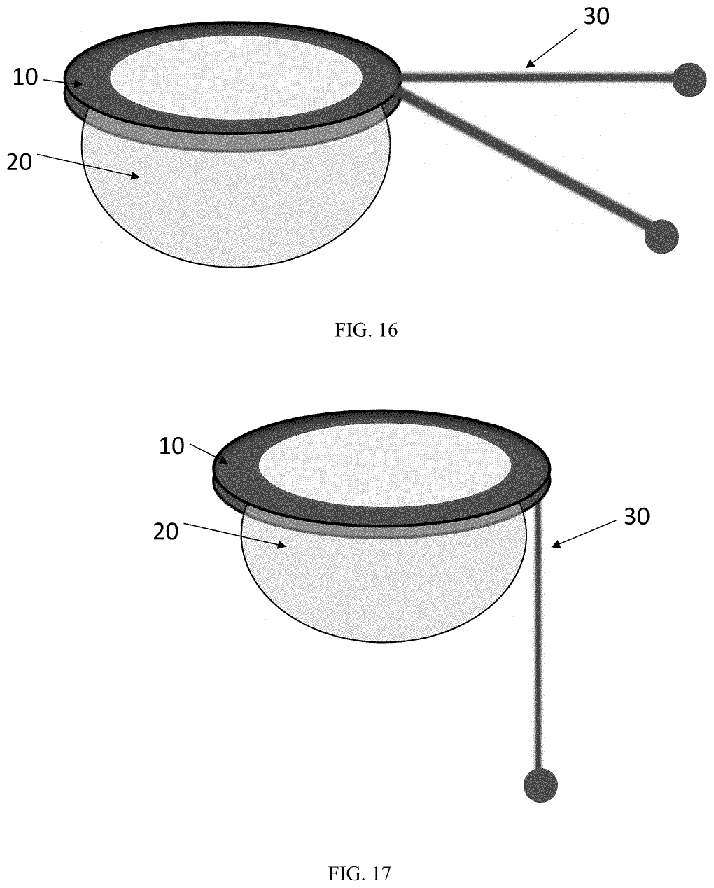

FIGS. 16 and 17 shown an embodiment in which the retrieval element 30, such as a string, is attached to the outer circumference surface of the ring 10. It is envisioned that attachment of the retrieval element 30 may be on the upper outer portion of the ring, the lower outer portion of the ring or anywhere in the outer part of the ring 10. Further, attachment of the retrieval element 30 can be made such that the retrieval element 30 extends from the ring 10 at varying angles.

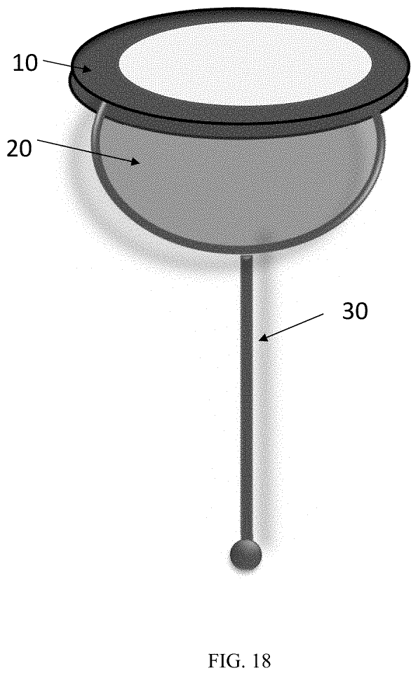

While it will be appreciated that the retrieval element 30 may be attached to the ring 10, the retrieval element 30 may alternatively be attached to the collection reservoir 20 as shown in FIGS. 18-20. FIGS. 18-20 show embodiments in which the membrane forming the collection reservoir 20 is reinforced for additional strength. As shown in the Figures, the retrieval element 30 is coupled to the collection reservoir 20 optionally via one or more reinforcing elements proximate or attached to the outer surface of the collection reservoir 20. In various aspects, the reinforcing elements may be coupled to the ring 10 as well as the retrieval element 30. In some aspects, the retrieval element 30 is attached to the outer portion of the collection reservoir 20 in a downward facing manner to facilitate removal.

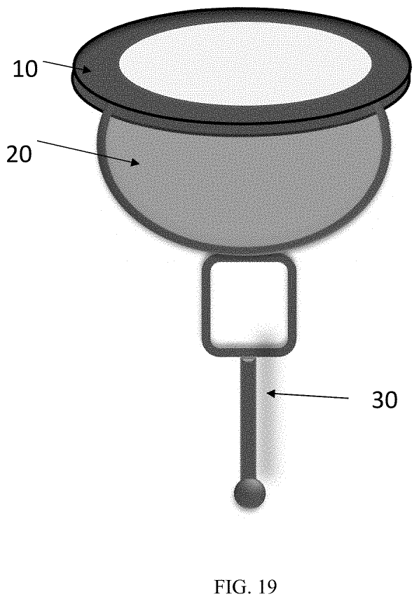

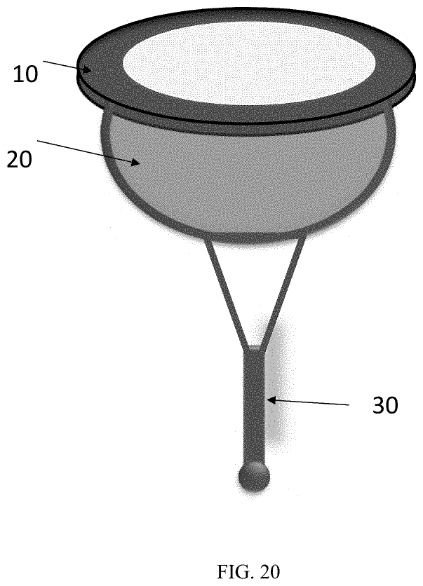

As discussed herein, the retrieval element may include one or more loops to facilitate removal by the user's finger. FIG. 19 shows an embodiment in which the retrieval element 30 includes a square shaped loop. FIG. 20 shows an embodiment in which the retrieval element 30 includes a triangular shaped loop. It is envisioned that the retrieval element 30 may include any number of loops of any geometric shape, including but not limited to circular, elliptical, square, triangular and the like.

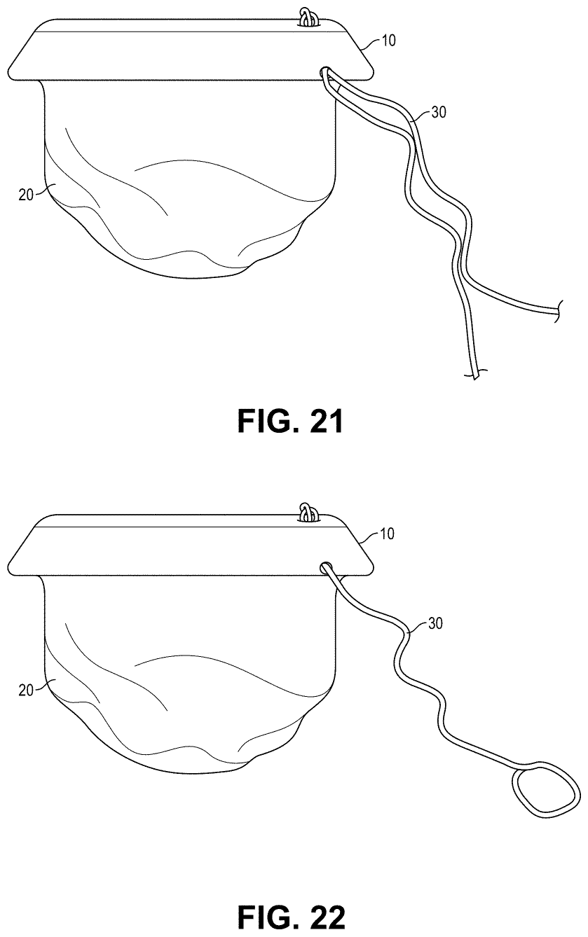

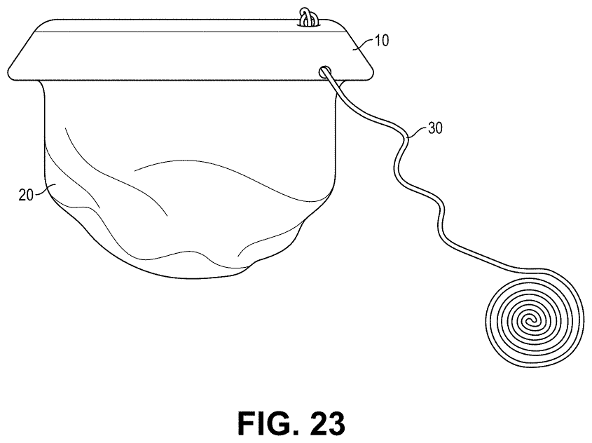

FIGS. 21-23 show embodiments of the device having different retrieval element configurations. FIG. 21 shows an embodiment in which the retrieval element 30 is a string that includes one or more knots to form one or more loops in the retrieval element 30. FIG. 22 shows an embodiment in which the retrieval element 30 is a string that includes a terminal loop to facilitate grasping. FIG. 23 shows an embodiment in which the retrieval element 30 is a string that includes a terminal ball or dot that facilitates grasping.

The retrieval element 30 may be composed of any biocompatible material. Additionally, the material of the retrieval element 30 is preferably hypoallergenic to prevent toxic shock syndrome (TSS).

As discussed herein, in various aspects, the retrieval element 30 is a string. Examples of various materials that may be utilized in forming a string include, but are not limited to cotton, Kevlar®, Vectran®, Spectra®, Dacron®, Dyneema®, Terlon® (PBT), Zylon® (PBO), polyimides (PIM), ultra-high molecular weight polyethylene, aramids, and the like.

Also provided is a kit which includes a collection device of the invention. The kit may also include a storage container for transport of the device containing an endocervical or vaginal sample, and optionally an applicator configured to deploy the device within the vaginal canal. In some aspects, the storage container includes a transport solution to enable safe ‘at home sampling’ of cervical cells originating from the cervical canal between 4 and 25 weeks of pregnancy. In some aspects, the storage container may be used without addition of any transport solution, or, alternatively, the container may contain a desiccant or other material to dry or otherwise preserve the sample. The kit described herein may be safely used by both healthcare professionals as well as individuals for specimen collection.

The disclosure also provides a method using the device of the disclosure which includes: a) disposing the collection device of the invention at a location proximal to an external os of a uterine cervix or anywhere within the vaginal canal of a subject; b) collecting an endocervical or vaginal sample in the collection reservoir while the device is located proximal to the external os or anywhere within the vaginal canal; c) removing the device containing the endocervical or vaginal sample from the subject; and optionally d) conducting an assay to detect an analyte present in the endocervical or vaginal sample.

The disclosure further provides a method of using the device of the invention which includes conducting an assay to detect an analyte present in an endocervical or vaginal sample from a subject, wherein the sample is collected into the device of the invention disposed at a location proximal to an external os of a uterine cervix or anywhere within the vaginal canal of the subject.

In embodiments, the endocervical or vaginal sample includes maternal cells and extravillous trophoblast cells or other cells of maternal, embryonic or extraembryonic origin from a pregnant or non-pregnant subject.

In embodiments, the method further includes isolating cells from the biological sample and analyzing DNA from the cells. Particular types of assays that may be performed to isolate and analyze the DNA in connection with the present disclosure are disclosed in WO 2017/176985, which is incorporated herein in its entirety.

In various aspects, the sample is obtained from a human female subject. In one aspect, the subject is pregnant. The pregnant subject maybe at least about 4 weeks, 5 weeks, 6 weeks, 7, weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 30 weeks, 35 weeks or 40 weeks of gestation.

In various aspects, the method and device of the invention allow a subject to easily collect a biological sample in a non-clinical setting, e.g., an at-home setting, which can be subsequently analyzed in a clinical setting. For example, once the sample is collected by the subject in an at-home setting, the sample is transported to a clinical setting for diagnostic analysis.

The method may further include conducting an assay to detect an analyte present in the sample. This may be performed once the reagent solution enters the sample reservoir. Alternatively, this may be performed by a clinician other than the user of the device at a remote clinical setting after transport of the device to the clinician.

In some aspects, analysis utilizes nucleic acids. However, it will be understood that the method of the invention may include analysis of any number of analytes which may be used to detect a disease or condition, such as a disease or condition associated with the subject and/or fetus of the subject. For example, an analyte may include, but not limited to, a cell or portion thereof, cellular nuclei or portion thereof, a biomolecule, such as a nucleic acid (e.g., DNA, RNA, mRNA, tRNA or miRNA), amino acid, protein, peptide, hormone, steroid, lipid, carbohydrate or ion, a chemical compound, such as a small organic compound, a microorganism or portion thereof (e.g., virus, bacteria or fungi). In one aspect, the analyte is human chorionic gonadotropin. Given the number of potential analyte targets, it is possible to detect a myriad of different conditions, diseases and/or disorders.

In certain aspects, analysis includes detecting a disease or condition associated with the subject or a developing fetus, including for example, cancer, sexually transmitted infection, pregnancy, fetal defect, status of microbiome or presence of one or more microorganisms.

The term “pregnancy-associated disorder,” as used herein, refers to any condition or disease that may affect a pregnant woman, the fetus the woman is carrying, or both the woman and the fetus. Such a condition or disease may manifest its symptoms during a limited time period, e.g., during pregnancy or delivery, or may last the entire life span of the fetus following its birth. Some examples of a pregnancy-associated disorder include ectopic pregnancy, preeclampsia, preterm labor, and fetal chromosomal abnormalities such as trisomy 13, 18, or 21.

The term “chromosomal abnormality” refers to a deviation between the structure of the subject chromosome and a normal homologous chromosome. The term “normal” refers to the predominate karyotype or banding pattern found in healthy individuals of a particular species. A chromosomal abnormality can be numerical or structural, and includes but is not limited to aneuploidy, polyploidy, inversion, a trisomy, a monosomy, duplication, deletion, deletion of a part of a chromosome, addition, addition of a part of chromosome, insertion, a fragment of a chromosome, a region of a chromosome, chromosomal rearrangement, and translocation. A chromosomal abnormality can be correlated with presence of a pathological condition or with a predisposition to develop a pathological condition.

Examples of fetal diseases or conditions resulting from genetic anomalies, gene mutations and chromosomal abnormalities include achondroplasia, Down syndrome, trisomy 21, trisomy 18, trisomy 13, Turner syndrome, Sickle cell disease, Cystic fibrosis, Fragile X syndrome, Muscular dystrophy (e.g., Duchenne muscular dystrophy), Tay-Sachs disease, Neural tube defects, such as spina bifida and anencephaly, Thalassemia, Polycystic kidney disease, Hemophilia A, Huntington's disease and congenital adrenal hyperplasia.

In certain aspects, analysis includes detecting a marker for female reproductive management, such as miscarriage, fetal growth restriction, preterm labor, ruptured membrane, or any combination thereof.

In certain aspects, analysis includes detecting a marker for female reproductive oncology, such as cancer, including but not limited to ovarian, uterine, or cervical cancer.

In certain aspects, analysis includes detecting a marker of a female reproductive disorder, such as endometriosis or polycystic ovarian syndrome.

In certain aspects, analysis includes detection of an infection, such as a cervical, vaginal or uterine infection.

In certain aspects, analysis includes detecting the presence and strain type of a virus, such as human papillomavirus.

Although the invention has been described with reference to the above examples, it will be understood that modifications and variations are encompassed within the spirit and scope of the invention. Accordingly, the invention is limited only by the following claims.

What is claimed is:

1. A device for collecting an endocervical or vaginal sample comprising:
a) a resiliently deformable ring having a central opening, wherein the ring has a thickness defined by an outer circumference surface and an inner circumference surface, and a height defined by an upper rim and a lower rim;
b) a flexible membrane coupled to the ring and covering the central opening thereby defining a collection reservoir; and
c) a retrieval element configured for removal of the device.

2. The device of claim 1, wherein the retrieval element is coupled to the deformable ring.

3. The device of claim 1, wherein the retrieval element is coupled to the flexible membrane.

4. The device of claim 1, wherein the deformable ring is approximately circular or oval in a non-deformed position.

5. The device of claim 1, wherein the deformable ring is sized to be disposed proximal to an external os of a uterine cervix or anywhere within the vaginal canal of a human subject.

6. The device of claim 1, wherein the ring and membrane are composed of a biocompatible polymer.

7. The device of claim 6, wherein the ring is composed of a first biocompatible polymer and the membrane is composed of a second biocompatible polymer.

8. The device of claim 6, wherein the ring and the membrane are composed of the same biocompatible polymer.

9. The device of claim 6, wherein the ring and membrane are composed of a non-silicon material.

10. The device of claim 1, wherein the retrieval element is composed of a hypoallergenic material.

11. The device of claim 1, wherein the retrieval element has a length of about 4-25 cm.

12. The device of claim 1, wherein the ring and flexible membrane are formed of a single unitary material.

13. The device of claim 12, wherein the ring, flexible membrane and retrieval element are formed of a single unitary material.

14. The device of claim 1, wherein the ring and the retrieval element are formed of a single unitary material.

15. A kit comprising the device of claim 1 and a container for transport of the device containing an endocervical or vaginal sample.

16. The kit of claim 15, further comprising an applicator configured to deploy the device within the vaginal canal.

17. A method comprising:
a) disposing the device of claim 1 at a location proximal to an external os of a uterine cervix or anywhere within the vaginal canal of a subject;
b) collecting an endocervical or vaginal sample in the collection reservoir while the device is located proximal to the external os or anywhere within the vaginal canal; and
c) removing the device containing the endocervical sample from the subject.

18. The method of claim 17, further comprising: d) conducting an assay to detect an analyte present in the endocervical or vaginal sample, wherein a)-c) are performed by the subject.

19. The method of claim 18, wherein a)-d) are performed by the subject.

20. The method of claim 18, wherein a)-c) are performed in an at-home setting and d) is performed by a clinician other than the subject in a clinical setting.

21. The method of claim 17, further comprising: wherein a)-c) are performed by a clinician.

22. The method of claim 21, further comprising: d) conducting an assay to detect an analyte present in the endocervical or vaginal sample.

23. The method of claim 22, wherein a)-d) are performed by the clinician.

24. A method comprising conducting an assay to detect an analyte present in an endocervical or vaginal sample from a subject, wherein the sample is collected into the device of claim 1 disposed at a location proximal to an external os of a uterine cervix or anywhere within the vaginal canal of the subject.
